(19) United States
(12) Patent Application Publication
(10) Pub. No.: US 2015/0282757 A1
(43) Pub. Date: Oct. 8, 2015

(54) HEALTH MANAGEMENT DEVICES AND METHODS

(71) Applicant: ABBOTT DIABETES CARE INC., Alameda, CA (US)

(72) Inventor: Marc B. Taub, Mountain View, CA (US)

(21) Appl. No.: 14/643,486

(22) Filed: Mar. 10, 2015

Related U.S. Application Data
(63) Continuation of application No. 12/143,725, filed on Jun. 20, 2008, now abandoned.
(60) Provisional application No. 60/945,578, filed on Jun. 21, 2007.

(51) Int. Cl. A61B 5/00; A61B 5/0205; A61B 5/11; A61B 5/145

(52) U.S. Cl. CPC A61B 5/486; A61B 5/14532; A61B 5/0205; A61B 5/4356; A61B 5/4866; A61B 5/1118; A61B 5/4848; A61B 5/4836; A61B 5/742; A61B 5/0004; A61B 5/0022; A61B 5/7465; A61B 5/14503; A61B 5/02411

(57) ABSTRACT

Methods, devices and systems to detect analyte level in a patient with gestational diabetes and/or provide related therapy management are provided.

### CROSS-REFERENCE TO RELATED APPLICATION

The present application is a continuation of U.S. non-provisional application Ser. No. 12/143,725 filed Jun. 20, 2008, which claims priority to U.S. provisional application No. 60/945,578 filed Jun. 21, 2007, both of which are incorporated by reference in their entirety and for all purposes.

### BACKGROUND

The detection of the level of analytes, such as glucose, lactate, oxygen, and the like, in certain individuals is vitally important to their health. For example, the monitoring of glucose is particularly important to individuals with diabetes. Diabetics may need to monitor glucose levels to determine when insulin is needed to reduce glucose levels in their bodies or when additional glucose is needed to raise the level of glucose in their bodies.

Accordingly, of interest are devices that allow a user to test for one or more analytes. Of particular interest are devices that may be used to monitor glucose levels, e.g., during particular times of increased risk for developing diabetes, e.g., before and/or during pregnancy.

### SUMMARY

Embodiments include analyte monitoring devices and methods, e.g., for glucose monitoring. Embodiments include continuous monitoring systems configured and used to detect or monitor one or more conditions associated with gestational diabetes, including detecting the onset and monitoring thereof.

Also provided are embodiments that include systems that enable glucose information to be downloaded from a continuous glucose system to a personal computer (“PC”) for user viewing and manipulation, and to generate reports. A health care provider may view the information remotely over a data network.

### BRIEF DESCRIPTION OF THE DRAWINGS

FIG. 1 shows a block diagram of an embodiment of a data monitoring and management system according to the present disclosure, e.g., to monitor glucose levels.

FIG. 2 shows a block diagram of an embodiment of the transmitter unit of the data monitoring and management system of FIG. 1.

FIG. 3 shows a block diagram of an embodiment of the receiver/monitor unit of the data monitoring and management system of FIG. 1.

FIG. 4 shows a schematic diagram of an embodiment of an analyte sensor according to the present disclosure.

FIGS. 5A-5B show a perspective view and a cross sectional view, respectively, of another embodiment an analyte sensor.

FIG. 6 shows an exemplary embodiment of a gestational diabetes monitoring system in accordance with one embodiment.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Generally, embodiments of the present disclosure relate to methods and devices for detecting at least one analyte, such as glucose, in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems and methods and/or transferring data between an in vivo continuous system and a BG meter system.

Accordingly, embodiments include analyte monitoring devices and systems that include an analyte sensor—at least a portion of which is positionable beneath the skin of the user—for the in vivo detection, of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a transmitter, receiver, transceiver, processor, etc. The sensor may be, for example, subcutaneously positionable in a patient for the continuous or periodic monitoring of a level of an analyte in a patient's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, which detected glucose may be used to infer the glucose level in the patient's bloodstream. Analyte sensors may be insertable into a vein, artery, or other portion of the body containing fluid. Embodiments of the analyte sensors of the subject disclosure may be configured for monitoring the level of the analyte over a time period which may range from minutes, hours, days, weeks, or longer. Analyte sensors that do not require bodily fluid contact are also contemplated.

Of interest are analyte sensors, such as glucose sensors, that are capable of in vivo detection of an analyte for about one hour or more, e.g., about a few hours or more, e.g., about a few days of more, e.g., about three or more days, e.g., about five days or more, e.g., about seven days or more, e.g., about several weeks or at least one month. Future analyte levels may be predicted based on information obtained, e.g., the current analyte level at time to, the rate of change of the analyte, etc. Predictive alarms may notify the user of a predicted analyte level that may be of concern in advance of the user's analyte level reaching the future level. This provides the user an opportunity to take corrective action.

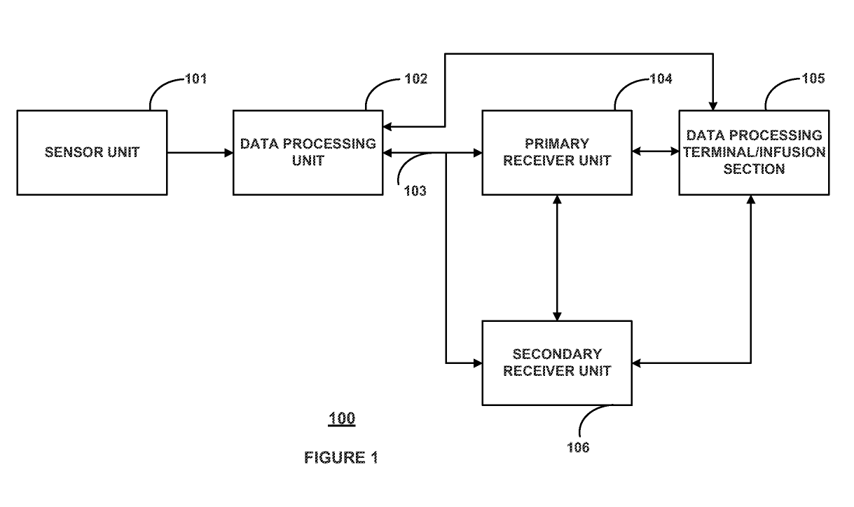

FIG. 1 shows a data monitoring and management system such as, for example, an analyte (e.g., glucose) monitoring system 100 in accordance with certain embodiments. Embodiments of the subject disclosure are further described primarily with respect to glucose monitoring devices and systems and methods of glucose detection, for convenience only, and such description is in no way intended to limit the scope of the disclosure. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

The analyte monitoring system 100 includes a sensor 101, a data processing unit 102 connectable to the sensor 101, and a primary receiver unit 104 which is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link which may optionally be configured for bi-directional communication. Further, the data processing unit 102 may include a transmitter or a transceiver to transmit and/or receive data to and/or from the primary receiver unit 104 and/or the data processing terminal 105 and/or optionally a secondary receiver unit 106.

Also shown in FIG. 1 is an optional secondary receiver unit 106 which is operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bi-directional wireless communication with each of the primary receiver unit 104 and the data processing terminal 105. As discussed in further detail below, in certain embodiments the secondary receiver unit 106 may be a de-featured receiver as compared to the primary receiver, i.e., the secondary receiver may include a limited or minimal number of functions and features as compared with the primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as the primary receiver unit 104. The secondary receiver unit 106 may include a docking portion to be mated with a docking cradle unit for placement by, e.g., the bedside for night time monitoring, and/or a bi-directional communication device. A docking cradle may recharge a power supply.

Only one sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous, semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to, at least periodically, sample the analyte level of the user and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element such as adhesive or the like to secure it to the user's body. A mount (not shown) attachable to the user and mateable with the unit 102 may be used. For example, a mount may include an adhesive surface. The data processing unit 102 performs data processing functions, where such functions may include but are not limited to, filtering and encoding of data signals, each of which corresponds to a sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In certain embodiments, the primary receiver unit 104 may include an analog interface section including and RF receiver and an antenna that is configured to communicate with the data processing unit 102 via the communication link 103, and a data processing section for processing the received data from the data processing unit 102 such as data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 1, the data processing terminal 105 may include a personal computer, a portable computer such as a laptop or a handheld device (e.g., personal digital assistant (PDA), telephone such as a cellular phone (e.g., a multimedia and Internet-enabled mobile phone such as an iPhone or similar phone), mp3 player, pager, and the like), or a drug delivery device, each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include an infusion device such as an insulin infusion pump or the like, which may be configured to administer insulin to patients, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device may be an external device, an internal device (wholly implantable in a user), or a partially implantable device.

In certain embodiments, the data processing terminal 105, which may include an insulin pump, may be configured to receive the analyte signals from the data processing unit 102, and thus, incorporate the functions of the primary receiver unit 104 including data processing for managing the patient's insulin therapy and analyte monitoring. In certain embodiments, the communication link 103 as well as one or more of the other communication interfaces shown in FIG. 1, may use one or more of: an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements), while avoiding potential data collision and interference.

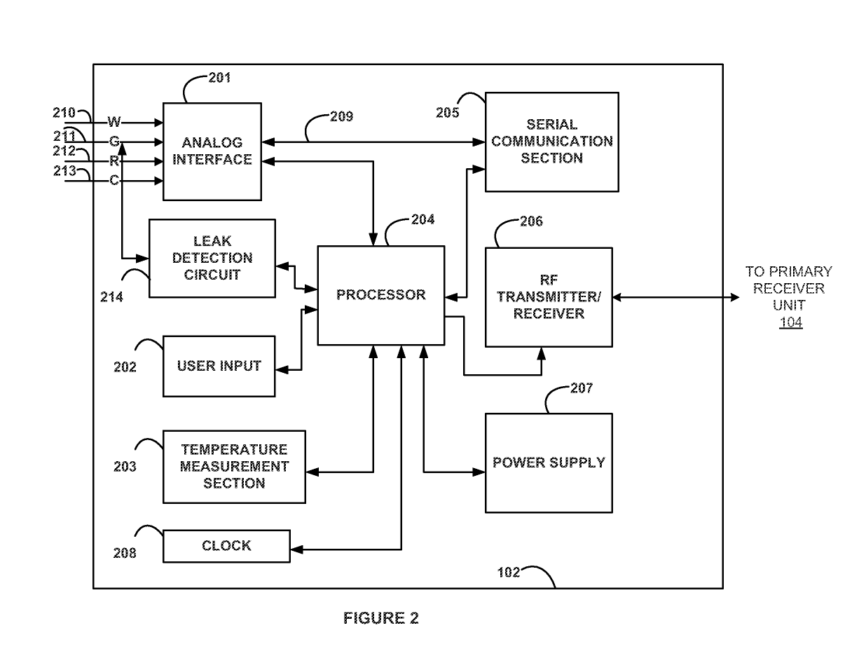

FIG. 2 shows a block diagram of an embodiment of a data processing unit of the data monitoring and detection system shown in FIG. 1. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using, for example, one or more state machines and buffers.

As can be seen in the embodiment of FIG. 2, the sensor unit 101 (FIG. 1) includes four contacts, three of which are electrodes—work electrode (W) 210, reference electrode (R) 212, and counter electrode (C) 213, each operatively coupled to the analog interface 201 of the data processing unit 102. This embodiment also shows an optional guard contact (G) 211. Fewer or greater electrodes may be employed. For example, the counter and reference electrode functions may be served by a single counter/reference electrode, or there may be more than one working electrode and/or reference electrode and/or counter electrode, etc.

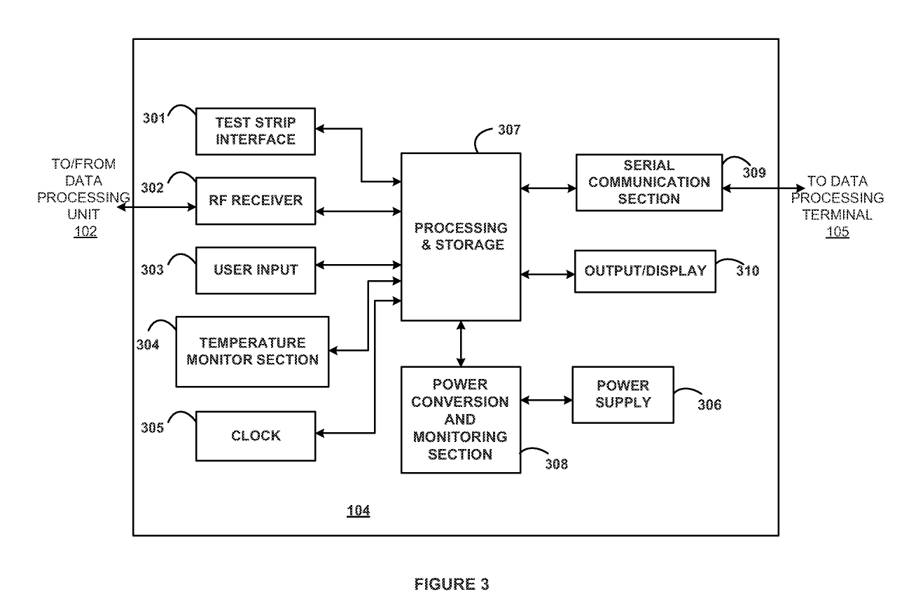

FIG. 3 is a block diagram of an embodiment of a receiver/monitor unit such as the primary receiver unit 104 of the data monitoring and management system shown in FIG. 1. The primary receiver unit 104 includes one or more of a blood glucose test strip interface 301, an RF receiver 302, an input 303, a temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the receiver processor 307. Moreover, also shown are a receiver serial communication section 309, and an output 310, each operatively coupled to the processing and storage unit 307. The receiver may include user input and/or interface components or may be free of user input and/or interface components.

In certain embodiments, the test strip interface 301 includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 310 of the primary receiver unit 104. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. FreeStyle® blood glucose test strips from Abbott Diabetes Care, Inc. Glucose information obtained by the in vitro glucose testing device may be used for a variety of purposes, computations, etc. For example, the information may be used to calibrate sensor 101, confirm results of the sensor 101 to increase the confidence thereof (e.g., in instances in which information obtained by sensor 101 is employed in therapy related decisions), etc.

In further embodiments, the data processing unit 102 and/or the primary receiver unit 104 and/or the secondary receiver unit 105, and/or the data processing terminal/infusion section 105 may be configured to receive the blood glucose value wirelessly over a communication link from, for example, a blood glucose meter. In further embodiments, a user manipulating or using the analyte monitoring system 100 (FIG. 1) may manually input the blood glucose value using, for example, a user interface (for example, a keyboard, keypad, voice commands, and the like) incorporated in the one or more of the data processing unit 102, the primary receiver unit 104, secondary receiver unit 106, or the data processing terminal/infusion section 105.

Additional detailed description of embodiments of test strips, blood glucose (BG) meters and continuous monitoring systems and data management systems that may be employed are provided in but not limited to: U.S. Pat. No. 6,175,752; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,120,676; U.S. Pat. No. 6,143,164; U.S. Pat. No. 6,299,757; U.S. Pat. No. 6,338,790; U.S. Pat. No. 6,377,894; U.S. Pat. No. 6,600,997; U.S. Pat. No. 6,773,671; U.S. Pat. No. 6,514,460; U.S. Pat. No. 6,592,745; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,299,757; U.S. Pat. No. 6,461,496; U.S. Pat. No. 6,503,381; U.S. Pat. No. 6,591,125; U.S. Pat. No. 6,616,819; U.S. Pat. No. 6,618,934; U.S. Pat. No. 6,676,816; U.S. Pat. No. 6,749,740; U.S. Pat. No. 6,893,545; U.S. Pat. No. 6,942,518; U.S. Pat. No. 6,514,718; U.S. patent application Ser. No. 10/745,878 filed Dec. 26, 2003 entitled “Continuous Glucose Monitoring System and Methods of Use”, and elsewhere, the disclosures of each which are incorporated herein by reference for all purposes.

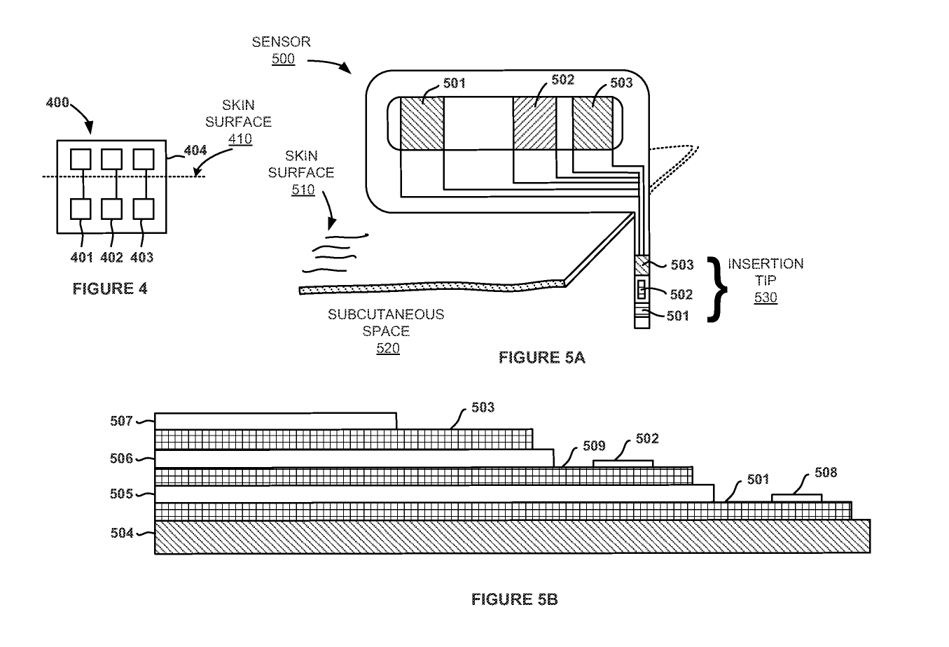

FIG. 4 schematically shows an embodiment of an analyte sensor in accordance with the present disclosure. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include but are not limited to aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor may be wholly implantable in a user or may be configured so that only a portion is positioned within (internal) a user and another portion outside (external) a user. For example, the sensor 400 may include a portion positionable above a surface of the skin 410, and a portion positioned below the skin. In such embodiments, the external portion may include contacts (connected to respective electrodes of the second portion by traces) to connect to another device also external to the user such as a transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

FIG. 5A shows a perspective view of an embodiment of an electrochemical analyte sensor 500 having a first portion (which in this embodiment may be characterized as a major portion) positionable above a surface of the skin 510, and a second portion (which in this embodiment may be characterized as a minor portion) that includes an insertion tip 530 positionable below the skin, e.g., penetrating through the skin and into, e.g., the subcutaneous space 520, in contact with the user's biofluid such as interstitial fluid. Contact portions of a working electrode 501, a reference electrode 502, and a counter electrode 503 are positioned on the portion of the sensor 500 situated above the skin surface 510. Working electrode 501, a reference electrode 502, and a counter electrode 503 are shown at the second section and particularly at the insertion tip 530. Traces may be provided from the electrode at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 510, 502 and 503, of the sensor 500 as well as the substrate and the dielectric layers are provided in a layered configuration or construction. For example, as shown in FIG. 5B, in one aspect, the sensor 500 (such as the sensor unit 101 FIG. 1), includes a substrate layer 504, and a first conducting layer 501 such as carbon, gold, etc., disposed on at least a portion of the substrate layer 504, and which may provide the working electrode. Also shown disposed on at least a portion of the first conducting layer 501 is a sensing layer 508.

A first insulation layer such as a first dielectric layer 505 is disposed or layered on at least a portion of the first conducting layer 501, and further, a second conducting layer 509 may be disposed or stacked on top of at least a portion of the first insulation layer (or dielectric layer) 505. As shown in FIG. 5B, the second conducting layer 509 may provide the reference electrode 502, and in one aspect, may include a layer of silver/silver chloride (Ag/AgCl), gold, etc.

A second insulation layer 506 such as a dielectric layer in one embodiment may be disposed or layered on at least a portion of the second conducting layer 509. Further, a third conducting layer 503 may provide the counter electrode 503. It may be disposed on at least a portion of the second insulation layer 506. Finally, a third insulation layer may be disposed or layered on at least a portion of the third conducting layer 503. In this manner, the sensor 500 may be layered such that at least a portion of each of the conducting layers is separated by a respective insulation layer (for example, a dielectric layer). The embodiment of FIGS. 5A and 5B show the layers having different lengths. Some or all of the layers may have the same or different lengths and/or widths.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing there between and/or include dielectric material or insulation material disposed between the conducting layers/electrodes. Furthermore, in certain embodiments one or more of the electrodes 501, 502, 503 may be disposed on opposing sides of the substrate 504. In such embodiments, contact pads may be one the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer (see for example sensing layer 408 of FIG. 5B) proximate to or on a surface of a working electrode. In many embodiments, a sensing layer is formed near or on only a small portion of at least a working electrode.

The sensing layer includes one or more components designed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided).

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, such as glucose oxidase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer 64 may be spaced apart from the working electrode, and separated from the working electrode, e.g., by a separation layer. A separation layer may include one or more membranes or films or a physical distance. In addition to separating the working electrode from the sensing layer the separation layer may also act as a mass transport limiting layer and/or an interferent eliminating layer and/or a biocompatible layer.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes such as ferrocene. Examples of inorganic redox species are hexacyanoferrate (III), ruthenium hexamine, etc.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, such as 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase or oligosaccharide dehydrogenase, flavine adenine dinucleotide (FAD) dependent glucose dehydrogenase, nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent (which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

Certain embodiments include a Wired Enzyme™ sensing layer (Abbott Diabetes Care, Inc.) that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is redox active component that includes (1) Osmium-based mediator molecules attached by stable (bidente) ligands anchored to a polymeric backbone, and (2) glucose oxidase enzyme molecules. These two constituents are cross-linked together.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e. reduction of the flux of analyte that can reach the sensing layer, (2) biocompatibility enhancement, or (3) interferent reduction.

Other sensors and sensor systems are contemplated as well. Such include, but are not limited to optical sensors, colorimetric sensors, potentiometric sensors, coulometric sensors, hydrogen peroxide detecting sensors, etc.

The description herein is directed primarily to electrochemical sensors for convenience only and is in no way intended to limit the scope of the disclosure. Other sensors and sensor systems are contemplated. Such include, but are not limited to, optical sensors, colorimetric sensors, and sensors that detect hydrogen peroxide to infer glucose levels, etc.

For example, a hydrogen peroxide-detecting sensor may be constructed in which a sensing layer includes enzyme such as glucose oxides, glucose dehydrogensae, or the like, and is positioned proximate to the working electrode. The sending layer may be covered by a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by crosslinking two components together, for example: (1) a redox compound such as a redox polymer containing pendent Os polypyridyl complexes with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by crosslinking together (1) a redox polymer containing pendent Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

A sensor may also include an active agent such as an anticlotting and/or antiglycolytic agent(s) disposed on at least a portion a sensor that is positioned in a user. An anticlotting agent may reduce or eliminate the clotting of blood or other body fluid around the sensor, particularly after insertion of the sensor. Examples of useful anticlotting agents include heparin and tissue plasminogen activator (TPA), as well as other known anticlotting agents. Embodiments may include an antiglycolytic agent or precursor thereof. Examples of antiglycolytic agents are glyceraldehyde, fluoride ion, and mannose.

Sensors may be configured to require no system calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, such as but not limited to glucose concentration and/or temperature and/or rate of change of glucose, etc.

Calibration may be accomplished using an in vitro test strip (or other reference), e.g., a small sample test strip such as a test strip that requires less than about 1 microliter of sample and/or has a short test e.g., such as Freestyle® or Precision® blood glucose monitoring systems available from Abbott Diabetes Care, Inc., of Alameda, Calif. (and the like). For example test strips that only require about 1 microliter or less sample, for example about 0.5 microliters or less, for example about 0.3 microliters or less, for example about 0.1 microliters or less. In some embodiments, the volume of sample may be as low as about 0.05 microliters or as low as about 0.03 microliters, in certain embodiments. Systems that have minimal test times may be used, e.g., test times may range from about 1 second to about 20 seconds, e.g., from about 3 seconds to about 10 seconds, e.g., from about 3 seconds to about 7 seconds, e.g., about 5 seconds or about 3 seconds, in certain embodiments.

In certain embodiments, a sensor may be calibrated using only one sample of body fluid per calibration event. For example, a user need only lance a body part one time to obtain sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Analyte monitoring systems may include an optional alarm system that, e.g., based on information from a processor, warns the patient of a potentially detrimental condition of the analyte. For example, if glucose is the analyte, an alarm system may warn a user of conditions such as hypoglycemia and/or hyperglycemia and/or impending hypoglycemia, and/or impending hyperglycemia. An alarm system may be triggered when analyte levels approach, reach or exceed a threshold value. An alarm system may also, or alternatively, be activated when the rate of change, or acceleration of the rate of change, in analyte level increase or decrease approaches, reaches or exceeds a threshold rate or acceleration. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

Embodiments include sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a data processing unit such as a transmitter, a receiver/display unit, and a data processing terminal/infusion section such as a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

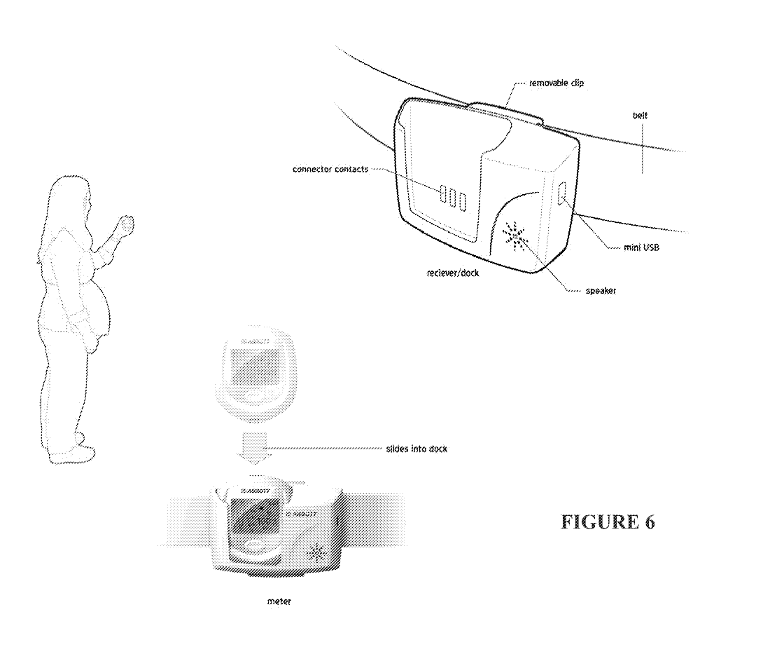

In certain embodiments, a pregnancy analyte monitoring system is employed to monitor (including to detect) gestational diabetes. For example, FIG. 6 shows an exemplary embodiment of a gestational diabetes monitoring system in accordance with one embodiment. As shown, the monitoring system may be configured as a belt holster to be worn around the waist during pregnancy. In one aspect, the receiver unit functionality of the analyte monitoring system may be integrated into the holster of the belt worn around the waist, and is configured to couple to a blood glucose meter or a other data processing unit via contacts of the holster.

The blood glucose meter (or other data processing unit) displays information to the user when electronically coupled to the holster, i.e., when docked or when in wireless signal communication with the belt holster (for example, when removed from the holster). The holster may include some or all functionality of a primary receiver unit as described below for continuous analyte monitoring. For example, the holster may contain some or all of a FreeStyle Navigator® system, e.g., the receiver functionality as described above. In one aspect, the belt holster may be configured such that the collected and stored analyte data may be transferred to the blood glucose meter when docked in the holster (or when wirelessly synchronized with the belt holster). The analyte monitoring system may be calibrated using the BG meter, e.g., when the blood glucose meter is docked.

A user and/or health care provider (“HCP”) may monitor a user's glucose levels prior to (e.g., in anticipation of) pregnancy and/or during pregnancy using an analyte monitoring system as described herein. Such embodiments will be herein referred to as “gestational diabetes” or “GD” systems. Applicability may be for the treatment of gestational diabetes, as well as the treatment of women with either Type 1 or Type 2 diabetes during pregnancy.

In certain embodiments, a GD system may be used in conjunction with (e.g., to confirm) a standard diagnostic diabetes test, e.g., a standard glucose tolerance test, or may be used instead of a standard test, i.e., may be the sole diagnostic test.

Embodiments may include assessing glucose tolerance of a pregnant woman, e.g., at about the 26^{th} week of pregnancy. If the assessment indicates gestational diabetic condition, or onset of such condition, lifestyle changes may be implemented, e.g., for one or more weeks, to try to control the diabetes using, for example, one or more of modification to the diet, administration of medication, exercise, and the like. During this period, glucose may be monitored using an in vitro blood glucose meter, e.g., a small volume (e.g., about 1 microliter or less) and/or short test time (e.g., about one to about 20 seconds, or less) BG system.

Embodiments include devices which allow diabetic patients to measure the blood (or other bodily fluid) glucose levels, e.g., hand-held electronic meters (blood glucose meters), e.g., such as Freestyle® or Precision® blood glucose monitoring systems available from Abbott Diabetes Care, Inc., of Alameda, Calif., which receives blood samples via enzyme-based test strips. Typically, a user inserts a test strip into a meter and lances a finger or alternate body site to obtain a blood sample. The drawn sample is applied to the test strip and the meter reads the strip and determines analyte concentration, which is then conveyed to the user. For example, the blood glucose meter converts a current generated by the enzymatic reaction in the test strip to a corresponding blood glucose value which is displayed or otherwise provided to the patient to show the level of glucose at the time of testing.

Such periodic discrete glucose testing helps diabetic patients to take any necessary corrective actions to better manage diabetic conditions.

Test strips may be adapted to measure the concentration of an analyte in any volume of sample, including but not limited to small volumes of sample, e.g., about 1 microliter or less sample, for example about 0.5 microliters or less, for example about 0.3 microliters or less, for example about 0.1 microliters or less. In some embodiments, the volume of sample may be as low as about 0.05 microliters or as low as about 0.03 microliters. Test strips may be short test time test strips. For example, test times may range from about 1 second to about 20 seconds, e.g., from about 3 seconds to about 10 seconds, e.g., from about 3 seconds to about 7 seconds, e.g., about 5 seconds or about 3 seconds.

Test strips may be configured so that an accurate analyte measurement may be obtained using a volume of sample that wholly or partially fills a sample chamber of a strip. In certain embodiments, a test may only start when sufficient sample has been applied to a test strip, e.g., as detected by a detector such as an electrode. A system may be programmed to allow re-application of additional sample if insufficient sample is firstly applied, e.g., the time to reapply sample may range from about 10 seconds to about 2 minutes, e.g., from about 30 seconds to about 60 seconds.

Test strips may be side fill, front fill, top fill or corner fill, or any combination thereof. Test strips may be calibration-free, e.g., minimal input (if any) is required of a user to calibrate. In certain embodiments, no calibration test strips may be employed. In such embodiments, the user need not take any action for calibration, i.e., calibration is invisible to a user.

Test strips are used with meters. In certain embodiments, meters may be integrated meters, i.e., a device which has at least one strip and at least a second element, such as a meter and/or a skin piercing element such as a lancet or the like, in the device. In some embodiments, a strip may be integrated with both a meter and a lancet, e.g., in a single housing. Having multiple elements together in one device reduces the number of devices needed to obtain an analyte level and facilitates the sampling process. For example, embodiments may include a housing that includes one or more analyte test strips, a skin piercing element and a processor for determining the concentration of an analyte in a sample applied to the strip. A plurality of strips may be retained in a magazine in the housing interior and, upon actuation by a user, a single strip may be dispensed from the magazine so that at least a portion extends out of the housing for use.

If diet and/or exercise do not effectively address the gestational diabetic condition, a GD system may be used. Specifically, in one aspect, an HCP may prescribe a GD system to be used by the patient. Glucose information obtained by the GD system may be reviewed at a remote site by the HCP, e.g., using a remote terminal or host terminal such as a server accessible to the user and the HCP over a data network. Data encryption/decryption, password protection, and other measures may be provided to protect the user's personal information as well as medical information communicated over the data network.

Embodiments may include data communication over a local area network, a wide area network, a metropolitan area network, over the internet and/or accessed by an internet browser, a dedicated secure network connection, using one or more of data communication protocols such as, for example, TCP/IP, https, wireless application protocol (WAP), IPv4 (Internet Protocol version 4), IPv6 (Internet Protocol version 6) and the like. Furthermore, data communication may include techniques for error detection and/or correction, data filtering and other data processing to ensure data integrity and/or validity.

Embodiments may include a fetal heart rate monitor coupled to or integrated with, e.g., the GD data processing unit and/or GD receiver unit (for example, one or more receiver units 104/106 of FIG. 1). Embodiments may also include an external uterine contraction monitor (e.g., tokodynamometer) into either the GD data processing unit and/or GD receiving unit. Such devices are generally used to monitor the duration, frequency, and relative pressure of uterine contractions with a transducer strapped to the maternal abdomen.

Embodiments of the present disclosure may be used by women with Type 1 or Type 2 diabetes who are hoping to become pregnant and include an ovulation predictor. For most women, temperature of 96 to 98 degrees is considered normal prior to ovulation and 97 to 99 degrees after ovulation. In certain embodiments, a temperature probe located on the skin such as on an on-body data processing unit may be used to monitor skin surface temperature (average or at some pre-selected or user-defined time). Alternatively, the temperature probe may be located on the analyte sensor or on an additional subcutaneously inserted probe. Software on the data receiver or other external devices or terminals may predict ovulation based upon this data.

Embodiments may include pregnancy-related data management software. For example, the CoPilot™ data management system from Abbott Diabetes Care, Inc., or the like, may be employed. With the data management software, it is possible to enhance the diet and exercise management, as well as track pregnancy progress—for example, counting down the days to the due date for delivery. Additionally, the data management software may be configured to perform daily/weekly information updates, for example, to provide information targeted to the current stage of pregnancy including physiological changes (such as, for example, signs and symptoms). The data management software may be further configured to track the development of the fetus by, for example, providing information on the growth and development of the fetus. Additionally, the calendaring function of the data management software may be used to conveniently track pregnancy milestones, as well as to track pregnancy related symptoms and/or complications. Additionally, the data management software may be used to provide prenatal care reminders and/recommendations, and also, provide pregnancy or birthing exercise tracking and/or recommendations.

For example, in one aspect, a user may be able to mark certain events in GD data (for example, by tagging or associating attributes or parameters to the GD data) to be viewed on the receiver once GD data is downloaded from the continuous monitoring system.

Patients with gestational diabetes are seen often by health care providers (for example approximately every 2 weeks). As such, logs, graphs, and reports may be tailored to this schedule and/or tailored to be appropriate to pregnancy stages/milestones. Moreover, the GD data may be specifically processed or otherwise mined in accordance with the pregnancy stages/milestones such that the logs, graphs and/or the reports may be customized for the particular pregnancy stage/milestone.

Target glycemic ranges are considerably narrower during pregnancy. Embodiments include GD systems that include such “modified ranges”. These may be reflected in logs, graphs, reports and alarms.

In certain embodiments, the analyte monitoring system may enable monitoring of more than one analyte, at the same or different times. For example, an analyte monitoring system may monitor glucose and ketones. This may be accomplished in vivo, or the analyte monitoring system may accept one or more test strips in one or more test strip ports (e.g., located on or coupled to a component of the system), the one or more test strips to determine glucose and ketones. Accordingly, a system may be configured to read each strip and determined the particular analyte concentration. In many embodiments, the system will know automatically which strip is inserted in the strip port (or such information may need to be entered).

For example, a strip may have a test strip type indicator such as a conductive element, memory element (e.g., on the strip or strip container, etc.), manually inputted code, and the like. In certain embodiments, an analyte monitoring system may include one test strip receiving port for receiving the different types of test strips, or may include separate strip receiving ports, each for a respective test strip type. Also contemplated are systems that receive strips from two or more manufacturers.

Accordingly, a method in one embodiment may include monitoring an analyte level of a subject with gestational diabetes over a predetermined time period, storing a plurality of data associated with the monitored analyte level over the predetermined time period, the plurality of data having one or more parameters associated with the monitored analyte level, and processing the stored plurality of data to determine, at least in part, one or more therapy regimen associated with the treatment of gestational diabetes.

A system for monitoring a glucose level of a patient with gestational diabetes in accordance with another embodiment includes an analyte sensor to monitor the analyte level of a patient with gestational diabetes over a predetermined time period, a data processing unit coupled to the analyte sensor, the data processing unit including a processor to process a plurality of signals associated with the detected analyte level, and a communication unit coupled to the data processing unit for communicating the plurality of signals associated with the detected analyte level of the patient to determine, at least in part, one or more therapy regimen associated with the treatment of gestational diabetes.

A method in one embodiment includes monitoring an analyte level of a subject for gestational diabetic related condition over a predetermined time period, storing a plurality of data associated with the monitored analyte level over the predetermined time period, the plurality of data having one or more parameters associated with the monitored analyte level, and processing the stored plurality of data to determine, at least in part, one or more therapy regimen associated with the treatment of gestational diabetes.

The method may include generating one or more output based on the processed stored plurality of data or the therapy regimen, where the one or more output includes one or more visual output or audible output.

The method may include providing the generated output to the subject, including displaying the generated output to the subject.

The one or more parameters may include one or more of the monitored analyte levels, fetal heart rate data, uterine contraction information, diet information, physical activity information, prenatal care information, or medication information.

The determined one or more therapy regimen may include a recommendation for modification to the diet of the subject, modification to the physical activity of the subject, or modification to the medication dosage information of the subject.

In one aspect, the determined therapy regimen includes modification to one or more of a modification to a basal rate profile for insulin delivery to the subject.

The analyte may be glucose.

A method in another aspect may include collecting analyte level information over a predetermined time period when one or more condition associated with gestational diabetes is detected, executing one or more computer program to process the collected analyte level information, wherein executing the one or more computer program includes: selecting a predetermined function associated with the detected gestational diabetes, retrieving one or more parameters associated with the collected analyte level information or the monitored gestational diabetes condition, performing data analysis based on the retrieved one or more parameters and the collected analyte level information to generate one or more therapy management information associated with the monitored one or more condition associated with gestational diabetes.

The one or more computer program may be executed on a healthcare provider computer terminal, or a patient computer terminal or a remote terminal.

The method may include transmitting the collected analyte level information to a remote location, where the analyte level information may be received over the internet.

In another aspect, the analyte level information may be encrypted when received, and in which case, the method may include decrypting the encrypted analyte information.

Also, the method may include storing the generated one or more therapy management information.

A system for monitoring glucose level of a patient with gestational diabetes in still another embodiment includes an analyte sensor to detect the analyte level of a patient with gestational diabetes over a predetermined time period, a data processing unit coupled to the analyte sensor, the data processing unit including a processor to process a plurality of signals associated with the detected analyte level, and a communication unit coupled to the data processing unit for communicating the plurality of signals associated with the detected analyte level of the patient to a remote location to determine, at least in part, one or more therapy regimen associated with the treatment of gestational diabetes.

The communication from the communication unit may be encrypted.

The remote location may include a computer terminal in communication with the communication unit, where the computer terminal may be configured to communicate with the communication unit over a wired or a wireless connection or both.

The remote location may include an output unit configured to output the one or more determined therapy regimen associated with the treatment of gestational diabetes.

Various other modifications and alterations in the structure and method of operation of the present disclosure will be apparent to those skilled in the art without departing from the scope and spirit of the present disclosure. Although the present disclosure has been described in connection with specific embodiments, it should be understood that the present disclosure as claimed should not be unduly limited to such specific embodiments. It is intended that the following claims define the scope of the present disclosure and that structures and methods within the scope of these claims and their equivalents be covered thereby.

What is claimed is:

1. A method, comprising:
monitoring an analyte level of a subject for gestational diabetic related condition over a predetermined time period;
storing a plurality of data associated with the monitored analyte level over the predetermined time period, the plurality of data having one or more parameters associated with the monitored analyte level; and
processing the stored plurality of data to determine, at least in part, one or more therapy regimen associated with the treatment of gestational diabetes.

2. The method of claim 1 generating one or more output based on the processed stored plurality of data or the therapy regimen.

3. The method of claim 2 wherein the one or more output includes one or more visual output or audible output.

4. The method of claim 2 including providing the generated output to the subject.

5. The method of claim 4 wherein providing the generated output includes displaying the generated output to the subject.

6. The method of claim 1 wherein the one or more parameters includes one or more of the monitored analyte levels, fetal heart rate data, uterine contraction information, diet information, physical activity information, prenatal care information, and medication information.

7. The method of claim 1 wherein the determined one or more therapy regimen includes a recommendation for modification to the diet of the subject, modification to the physical activity of the subject, or modification to the medication dosage information of the subject.

8. The method of claim 1 wherein the determined therapy regimen includes modification to one or more of a modification to a basal rate profile for insulin delivery to the subject.

9. The method of claim 1 wherein the analyte is glucose.

10. A method, comprising:
collecting analyte level information over a predetermined time period when one or more condition associated with gestational diabetes is detected; and
executing one or more computer program to process the collected analyte level information, wherein executing the one or more computer program includes:
selecting a predetermined function associated with the detected gestational diabetes;
retrieving one or more parameters associated with the collected analyte level information or the monitored gestational diabetes condition;
performing data analysis based on the retrieved one or more parameters and the collected analyte level information to generate one or more therapy management information associated with the monitored one or more condition associated with gestational diabetes.

11. The method of claim 10 wherein the one or more computer program is executed on a healthcare provider computer terminal, or a patient computer terminal or a remote terminal.

12. The method of claim 10 including transmitting the collected analyte level information to a remote location.

13. The method of claim 12 wherein the analyte level information is received over the internet.

14. The method of claim 13 wherein the analyte level information is encrypted when received.

15. The method of claim 14 including decrypting the encrypted analyte information.

16. The method of claim 10 including storing the generated one or more therapy management information.

17. A system for monitoring glucose level of a patient with gestational diabetes, comprising:
an analyte sensor to detect the analyte level of a patient with gestational diabetes over a predetermined time period;
a data processing unit coupled to the analyte sensor, the data processing unit including a processor to process a plurality of signals associated with the detected analyte level; and
a communication unit coupled to the data processing unit for communicating the plurality of signals associated with the detected analyte level of the patient to a remote location to determine, at least in part, one or more therapy regimen associated with the treatment of gestational diabetes.

18. The system of claim 17 wherein the communication from the communication unit is encrypted.

19. The system of claim 17 wherein the remote location includes a computer terminal in communication with the communication unit.

20. The system of claim 19 wherein the computer terminal is configured to communicate with the communication unit over a wired or a wireless connection or both.
